(19) United States
(12) Patent Application Publication
(10) Pub. No.: US 2025/0160353 A1
(43) Pub. Date: May 22, 2025

(54) CO-PARTICULATED MILK PROTEINS AND METHODS OF MAKING THEM

(71) Applicant: Leprino Foods Company, Denver, CO (US)

(72) Inventors: Richard K Merrill, Highlands Ranch, CO (US); Paulos Tesfe, Commerce City, CO (US); Jiancai Li, Englewood, CO (US); Chatrapati Veera Raghava Kumar Tammineedi, Broomfield, CO (US)

(73) Assignee: Leprino Foods Company, Denver, CO (US)

(21) Appl. No.: 18/655,042

(22) Filed: May 3, 2024

Related U.S. Application Data
(60) Provisional application No. 63/602,002, filed on Nov. 22, 2023.

(51) Int. Cl. A23C 9/152; A23C 9/13; A23C 21/06; A23J 3/10; C12N 9/52

(52) U.S. Cl. CPC A23C 9/1526; A23C 9/1307; A23C 21/06; A23J 3/10; C12N 9/52; C12Y 304/21

(57) ABSTRACT

The present disclosure includes co-particulated milk protein compositions. The compositions include at least 60 wt. % total protein on a dry weight basis, and a blend of whey proteins and casein protein. Compositions include where at least a portion of the total true protein includes casein-whey protein complexes. Compositions include where the total protein includes milk protein particles, where the milk protein particles having a particle size of 1 to 10 μm form less than 50 wt. % of the total true protein. Compositions include where the co-particulated milk protein composition exhibits a co-particulated index of greater than zero to less than or about 0.5%.

### CROSS-REFERENCE TO RELATED APPLICATIONS

This application claims benefit of U.S. Provisional Patent Application No. 63/602,002, entitled “CO-PARTICULATED MILK PROTEINS AND METHODS OF MAKING THEM,” filed Nov. 22, 2023, the contents of which are hereby incorporated by reference in their entirety.

### FIELD

The present technology relates to milk protein compositions, including particulated milk proteins having casein-whey complexes.

### BACKGROUND

Powdered milk-derived products such as milk proteins, aldobionic products, and galactooligosaccharides, have become a major source of ingredients in a wide variety of foods and beverages. Milk-derived proteins, for example, have become a major source of protein-fortification in nutrition bars, sports drinks, and yogurt products. One source of milk proteins is the whey protein that is produced as a byproduct of cheese making. During cheesemaking, the casein proteins in the milk are formed into cheese curds while the liquid whey is drained from the curds and diverted for further processing. In most cheesemaking processes, the liquid whey is a mixture of whey proteins and a significant amount of lactose and minerals, and the mixture undergoes additional purification to separate the whey proteins from the lactose and minerals.

Whey proteins derived from cheesemaking also include additional byproducts, such as cheesemaking enzymes and the hydrolyzed proteins they generate. The native glycomacropeptides (GMPs) are considered an inferior source of protein for muscle recovery because they have fewer branched chain amino acids, especially leucine, that stimulate muscle protein synthesis and are a major building block in muscle tissue following periods of intense exercise and resistance training. In addition, the native GMP content in whey protein compositions, as well as the particle size and agglomerate composition of native whey proteins and microparticulated whey proteins, can lead to undesirable flavor and poor process incorporation such as unacceptably high viscosity, reducing the total amount of protein capable of being fortified into the product. These and other challenges are addressed by the present technology.

### BRIEF SUMMARY

The present technology is generally directed to co-particulated milk protein compositions. Co-particulated milk protein compositions include at least 60 wt. % total protein on a dry weight basis, based upon the weight of the co-particulated milk protein composition. Co-particulated milk protein compositions include greater than about 60 wt. % whey protein based on the weight of total true protein and from about 5 wt. % to about 40 wt. % casein protein based on the weight of total true protein. Co-particulated milk protein compositions include where at least a portion of the total protein includes casein-whey protein complexes. Compositions include where the co-particulated milk protein composition exhibits a co-particulated index of greater than zero to less than or about 0.5%.

In embodiments, co-particulated milk protein compositions include milk protein particles having a particle size of 1 to 10 μm, where the milk protein particles having a particle size of 1 to 10 μm form less than 50 wt. % of the total true protein. In more embodiments, the whey protein and the micellar casein protein are present in the co-particulated milk protein composition at a ratio of whey protein to micellar casein protein of about 60:40 to about 95:5. Furthermore, in embodiments, the co-particulated milk protein compositions include less than or about 10 wt. % fat on a dry weight basis, based upon the weight of the co-particulated milk protein composition. Additionally or alternatively, in embodiments, the co-particulated milk protein composition includes milk protein particles, wherein milk protein particles having a particle size of less than 0.5 μm form greater than or about 30 wt. % of the total true protein. In yet more embodiments, the casein-whey protein complexes include one or more covalent disulfide bonds.

The present technology is also generally directed to co-particulated milk protein compositions that include at least 60 wt. % total protein on a dry weight basis, based upon the weight of the co-particulated milk protein composition. Co-particulated milk protein compositions include greater than about 60 wt. % whey protein based on the weight of total true protein and from about 5 wt. % to about 40 wt. % casein protein based on the weight of total true protein. Co-particulated milk protein compositions include milk protein particles having a particle size of 1 to 10 μm, where the milk protein particles having a particle size of 1 to 10 μm form less than 50 wt. % of the total true protein.

In embodiments, the co-particulated milk protein composition includes milk protein particles, wherein milk protein particles having a particle size of less than 0.5 μm form greater than or about 30 wt. % of the total true protein. In more embodiments, milk protein particles having a particle size of 1 to 10 μm form less than 45 wt. % of the total true protein. Furthermore, in embodiments, milk protein particles having a particle size of 1 to 10 μm form less than 40 wt. % of the total true protein. Additionally or alternatively, the co-particulated milk protein compositions include greater than or about 7 wt. % alpha-lactalbumin on a dry weight basis, based on the weight of total true protein.

In embodiments, the present technology is generally directed to fortified food and/or beverage products. Fortified food and/or beverage products include greater than or about 4 wt. % protein, based upon a weight of the food and/or beverage product. Fortified food and/or beverage products include a co-particulated milk protein composition according to any one or more of the embodiments discussed herein. For instance, in embodiments, fortified food and/or beverage compositions may include co-particulated milk protein compositions include at least 60 wt. % total protein on a dry weight basis, based upon the weight of the co-particulated milk protein composition, greater than about 60 wt. % whey protein based on the weight of total true protein and from about 5 wt. % to about 40 wt. % casein protein based on the weight of total true protein, and that include where at least a portion of the total protein includes casein-whey protein complexes. In embodiments, fortified food and/or beverage compositions may include co-particulated milk protein compositions that include at least 60 wt. % total protein on a dry weight basis, based upon the weight of the co-particulated milk protein composition, greater than about 60 wt. % whey protein based on the weight of total true protein, from about 5 wt. % to about 40 wt. % casein protein based on the weight of total true protein, and milk protein particles having a particle size of 1 to 10 μm, where the milk protein particles having a particle size of 1 to 10 μm form less than 50 wt. % of the total true protein. In embodiments, the fortified food and/or beverage product includes a spoonable or drinkable yoghurt.

The present technology is also generally directed to methods of making co-particulated milk protein compositions. Methods include forming a mixture of a whey protein composition and a casein source at a ratio of about 60:40 to about 95:5. Methods include denaturing the mixture at a temperature of greater than or about 70° C. and a pH of less than or about 6.8, forming co-particulated milk protein particles. Methods include reducing a particle size of the co-particulated milk protein particles in the mixture, thereby forming the co-particulated milk protein composition. Methods include where the co-particulated milk protein composition includes at least 60 wt. % total protein on a dry weight basis, based upon the weight of the co-particulated milk protein composition. Methods include where the co-particulated milk protein particles include milk protein particles having a particle size of 1 to 10 μm, and where the milk protein particles having a particle size of 1 to 10 μm form less than 50 wt. % of the total true protein.

In embodiments, the casein source includes micellar casein concentrate, micellar casein isolate, ultrafiltered milk protein, microfiltered milk protein, milk protein concentrate, milk protein isolate, a caseinate, a caseinate salt, rennet casein, acid casein, non-animal casein, or a combination thereof. In more embodiments, the whey protein composition includes whey protein concentrate, whey protein isolate, native whey protein concentrate, native whey protein isolate, acid whey protein, or a combination thereof. In further embodiments, denaturing includes forming one or more covalent disulfide bonds between a casein and a whey protein. The present technology is also generally directed to a fortified food and/or beverage product formed according to any one or more of the above methods.

### BRIEF DESCRIPTION OF THE DRAWINGS

A further understanding of the nature and advantages of selected embodiments of the present technology may be realized by reference to the remaining portions of the specification and the drawings wherein like reference numerals may be used throughout the several drawings to refer to similar components. In some instances, a sublabel is associated with a reference numeral and follows a hyphen to denote one of multiple similar components. When reference is made to a reference numeral without specification to an existing sublabel, it is intended to refer to all such multiple similar components.

FIG. 1 shows selected operations in a formation method according to some embodiments of the present technology.

FIG. 2A is a graph illustrating the particle size distribution of various feed materials.

FIG. 2B is a graph illustrating the particle size distribution of thermally treated feed materials.

FIG. 2C is a chart illustrating a comparison of particle size of a co-particulated milk protein composition according to embodiments of the present technology, an untreated feed material, and a microparticulated whey protein composition.

FIG. 3 is a chart illustrating the percentage of milk protein particles having particular size ranges in examples discussed herein.

FIG. 4 is a chart illustrating yogurt fermentation time according to examples of the present technology.

FIG. 5 is a chart illustrating viscosity of drinkable yogurts formed according to examples of the present technology.

FIG. 6 is a chart illustrating viscosity of spoonable yogurts formed according to examples of the present technology.

FIG. 7 is a chart illustrating percentages of particles anticipated to be present in samples formed according to examples of the present technology.

FIG. 8A is a chart illustrating % Kappa-casein in examples according to the present technology.

FIG. 8B is a chart illustrating an aggregation index of examples according to the present technology.

Several of the figures are included as schematics. It is to be understood that the figures are for illustrative purposes, and are not to be considered of scale unless specifically stated to be of scale. Additionally, as schematics, the figures are provided to aid comprehension and may not include all aspects or information compared to realistic representations, and may include exaggerated material for illustrative purposes.

In the figures, similar components and/or features may have the same numerical reference label. Further, various components of the same type may be distinguished by following the reference label by a letter that distinguishes among the similar components and/or features. If only the first numerical reference label is used in the specification, the description is applicable to any one of the similar components and/or features having the same first numerical reference label irrespective of the letter suffix.

### DETAILED DESCRIPTION

“Whey protein”, as referred to herein, is a collection of different proteins that primarily include β-lactoglobulin (β-Lg) and α-lactalbumin (α-La), bovine serum albumin, lactoferrin, lactoperoxidase, immunoglobulins, as well as glycomacropeptides (GMPs, sometimes referred to as cGMP or CMP) cleaved by chymosin activity from the native kappa-casein protein in the milk resulting in milk coagulation into the cheese curd. Depending on the purification process and the extent of purification, a whey protein concentrate (WPC) may be formed through concentration of the whey protein to 25-90 wt. % of protein as a percentage of the total weight of solids, or whey protein isolate (WPI) may be formed through the concentration of the whey proteins to 90-99 wt. % of protein as a percentage of the total weight of solids. Alternatively, whey may be extracted directly from milk as the permeate of a microfiltration process without cheese making. In this circumstance no GMP would be found in the native whey protein concentrate or native whey protein isolate because cheese was not made from the initial milk. The retentate of the microfiltration contains primarily micellar casein that is produced as a micellar casein concentrate or micellar casein isolate. Yet another source of whey is acid whey that is formed from the acid precipitation of casein from milk.

Whey proteins derived from cheesemaking also include additional byproducts, such as cheesemaking enzymes and the hydrolyzed proteins they generate. For instance, conventional compositions of whey obtained from enzymatically coagulated Bovine milk* typically contain:

| Beta- | Alpha- | Ratio of | | Total | Ratio of GMP |
|---|---|---|---|---|---|
| lactoglobulin | lactalbumin | Beta-lg to | Glycomacropeptide | Protein | to Total |
| (wt. %) | (wt. %) | Alpha-lg | (wt. %) | (WPC) | Protein |
| 50-60 | 12-16 | 3.13 to 5.00 | 15-21 | 25-80 | 0.19 to 1.00 |

* 1 Walstra P, Wouters J T M, Geurts T J. Milk Components, Dairy Science and Technology. 2nd ed. CRC Press; 2006: Chapter 2.
2 Foegeding E A, Luck P, Vardhanabhuti B. Encyclopedia of Dairy Sciences. 2nd ed. Elsevier Ltd.; 2011: Whey Protein Products.

The hydrolyzed proteins include glycomacropeptides (GMPs) that are hydrolyzed from k-casein so that the resulting para-K-casein can form a major component of cheese curd. The smaller, more soluble GMPs are carried away with the whey proteins and can constitute 13-20 wt. % of the protein present in the whey protein fraction on a dry weight basis.

“Casein” as referred to herein includes a collection of different proteins that primarily include αₛ₁-casein, αₛ₂-casein, β-casein, γ-casein, and κ-casein, and may be in micellar form, referred to as micellar casein or milk protein concentrate. As used herein, “micellar casein” includes a collection of all casein proteins in a micellar form. As used herein, “milk” includes a collection of all casein proteins and whey proteins in the ratio naturally found in milk of 80% casein and 20% whey proteins. Conversely, some protein sources, such as native whey may include β-casein. Caseins such as αₛ₂-casein, γ-casein, and κ-casein may contain one or more cysteine residues (e.g., one or more sulfur containing residues) that may interact with whey proteins that contain cysteine residues during thermal denaturation, whereas αₛ₁-casein and β-casein do not contain cysteine residues and hence do not interact with whey proteins during thermal denaturation.

Namely, thermal denaturation is generally conducted in the temperature range of 70-100° C. At such denaturation temperatures, no discernible structural alterations are observed in the casein micelle of milk proteins. However, the whey protein component of milk, such as bovine whey protein, primarily composed of β-lactoglobulin (β-lg) and α-lactalbumin (α-lac), does undergo significant changes upon heating at thermal denaturation temperatures. As the temperature rises, β-lg undergoes conformational changes, exposing a reactive thiol group. This exposed thiol group can create disulfide connections with other proteins possessing reactive thiol groups or engage in thiol group-disulfide bridge exchange reactions. At denaturation temperatures, such exchange reactions form covalent thiol group-disulfide bridges, rendering the denaturation process irreversible.

Due to the absence of a free thiol group, α-Lac cannot initiate polymerization. Nevertheless, it becomes irreversibly denatured in the presence of β-lg, which has four disulfide bridges, owing to thiol group-disulfide bridge exchange reactions. Additionally, β-lg engages in interactions with casein micelles through similar thiol group-disulfide bridge exchange reactions with κ- and αₛ₂-casein. While the initial phase of this process is believed to be physical in nature, the ultimate interaction is covalent, specifically disulfide-linked. Therefore, following heating, the casein micelles' “hairy layer” contains whey proteins associated with casein. However, not all β-lg and α-lac become bound to the casein micelle, a significant portion forms pure whey protein aggregates. Thus, heat treatment (e.g. thermal denaturation) of milk proteins that include cysteine residue-containing casein proteins leads to a complex mixture that includes native whey proteins, whey protein aggregates, and casein micelles enveloped in whey protein.

The present technology overcomes issues of high viscosity and increased fermentation time of fermented foods, such as yogurt preparations, as well as other problems, by providing a particulated milk protein composition containing a tailored blend of whey proteins and casein proteins. Namely, the present technology has surprisingly found that by blending one or more whey proteins with cysteine residue-containing casein proteins, such as casein micelles, prior to particulation, the co-particulated milk protein composition exhibits a unique composition of casein-whey protein aggregates and whey-whey aggregates. Such aggregates surprisingly contribute to a low viscosity in fortified foods. In addition, the casein-whey protein aggregates exhibit smaller particle sizes than traditional particulated whey protein compositions, which may further contribute to the low viscosity increase exhibited when incorporated into a food and/or beverage product, even in highly fortified foods and/or beverages. It was also surprisingly discovery that co-particulated milk protein compositions significantly reduce the fermentation time of yogurts as compared to a yogurt containing microparticulated whey protein without co-particulated non-whey milk proteins.

For instance, compositions according to one or more embodiments of the present disclosure may exhibit lower process viscosity and decreased fermentation time, which exhibiting excellent taste and sensory properties. Namely, the present disclosure has surprisingly found that when co-particulated milk proteins are utilized, there is a reduction in process viscosity such that the viscosity is about 10% less than a viscosity of a composition fortified with a microparticulated whey protein, such about 20% less, such as about 30% less, such as about 40% less, such as about 50% less than a viscosity of a non-enzymatically reduced microparticulated whey protein composition. For instance, in a microparticulated whey protein composition that has not been co-particulated as discussed herein, the process viscosity of the composition may be greater than 200 centipoise, such as from 200 centipoise to 500 centipoise. Conversely, compositions according to the present technology exhibit a process viscosity of less than 200 centipoise, such as about 175 centipoise or less, such as about 150 centipoise or less, such as about 125 centipoise or less, such as about 100 centipoise or less, such as about 75 centipoise or less, such as about 50 centipoise or less, such as down to about 25 centipoise, or any ranges or values therebetween. In some embodiments, compositions according to the present technology may also have reduced ability to bind water. Thus, compositions according to the present technology may be uniquely suited to fortify water-containing foods to levels higher than previously believed possible with whey protein compositions, as there is lower observed viscosity increases typically associated with protein compositions utilized to fortify food and/or beverage formulations.

Moreover, co-particulated milk protein compositions have surprisingly been found to decrease fermentation time without change in temperature or starter culture. Thus, in embodiments, a fermented food and/or beverage fortified utilizing a co-particulated milk protein composition according to the present technology may exhibit a fermentation time that is greater than or about 5% faster than a food and/or beverage fortified to the same level with a microparticulated whey protein composition, such as greater than or about 7.5%, such as greater than or about 10%, such as greater than or about 12.5%, such as greater than or about 15%, such as greater than or about 17.5%, such as greater than or about 20% faster, or any ranges or values therebetween.

For instance, in embodiments, the co-particulated milk protein composition may contain greater than or about 5 wt. % casein-whey aggregates, based on the weight of total true protein, such as greater than or about 10 wt. %, such as greater than or about 15 wt. %, such as greater than or about 20 wt. %, such as greater than or about 25 wt. %, such as greater than or about 30 wt. %, such as greater than or about 35 wt. %, such as greater than or about 40 wt. %, such as greater than or about 45 wt. %, such as greater than or about 50 wt. %, such as greater than or about 55 wt. %, such as greater than or about 60 wt. % casein-whey aggregates based on the weight of total true protein, or any ranges or values therebetween. As discussed herein, casein-whey aggregates may also be referred to as casein-whey complexes due to the covalent bonds formed between casein and whey protein particles. Namely, as discussed above, the casein proteins utilized herein may contain one or more cysteine containing-residues capable of covalently bonding with one or more whey proteins.

Stated differently, the co-particulated milk protein composition may have a co-particulation index (measured according to the examples below) of greater than zero up to about 1%, such as greater than or about 0.05%, greater than or about 0.1%, greater than or about 0.15%, greater than or about 0.2%, greater than or about 0.25%, greater than or about 0.3%, greater than or about 0.35%, greater than or about 0.4%, greater than or about 0.45%, greater than or about 0.5%, greater than or about 0.55%, greater than or about 0.6%, or such as less than or about 0.9%, less than or about 0.8%, less than or about 0.7%, less than or about 0.6%, less than or about 0.5%, less than or about 0.4%, less than or about 0.3%, less than or about 0.2%, or any ranges or values therebetween.

Surprisingly, the protein particles present in the co-particulated milk protein composition also exhibit an advantageous particle size. Namely, co-particulated milk protein particles having a size of from 1 μm to 10 μm may form less than 45 wt. % of the total true protein, such as less than or about 44 wt. %, such as less than or about 43 wt. %, such as less than or about 42 wt. %, such as less than or about 41 wt. %, such as less than or about 40 wt. %, of the total true protein, or any ranges or values therebetween. Furthermore, co-particulated milk protein particles having a size of from 0.5 μm to 1.5 μm may form less than or about 60 wt. % of the total true protein, such as less than or about 57.5 wt. %, such as less than or about 55 wt. %, such as less than or about 52.5 wt. %, such as less than or about 50 wt. % of the total true protein, or any ranges or values therebetween. In embodiments, co-particulated milk protein particles having a size of less than or about 0.5 μm may form greater than or about 20 wt. % of the total true protein, such as greater than or about 22.5 wt. %, such as greater than or about 25 wt. %, such as greater than or about 27.5 wt. %, such as greater than or about 30 wt. %, such as greater than or about 35 wt. %, such as greater than or about 40 wt. %, such as greater than or about 45 wt. %, such as greater than or about 50 wt. %, such as greater than or about 55 wt. %, such as greater than or about 60 wt. % of the total true protein, or any ranges or values therebetween.

Nonetheless, the particles of the co-particulated milk protein composition according to the present technology can have an average particle diameter of about 0.001 μm to about 11 μm, such as about 0.005 μm to about 9 μm, such as about 0.01 μm to about 7 μm, such as about 0.015 μm to about 5 μm, or any ranges or values therebetween.

Surprisingly, the present technology has found that by forming a co-particulated milk protein composition as discussed herein, a narrow particle size distribution of the co-particulated milk protein particles can be obtained, which can further improve the flavor attributes, viscosity properties, and fermentation time of products incorporating the co-particulated milk protein composition of the present technology. For instance, the particles can have a D90 particle size distribution value, which is the particle diameter where 90% of the sample's mass includes particles of that size or smaller, of about 5 μm or less, such as about 4.5 μm or less, such as about 4 μm or less, such as about 3.5 μm or less, such as about 3 μm or less, such as about 2.5 μm or less, such as about 2 μm or less, or any ranges or values therebetween.

Furthermore, the particles can have a D50 particle size distribution value, which is the particle diameter where 50% of the sample's mass includes particles of that size or smaller, of about 5 μm or less, such as about 4.5 μm or less, such as about 4 μm or less, such as about 3.5 μm or less, such as about 3 μm or less, such as about 2.5 μm or less, such as about 2 μm or less, such as about 1.5 μm or less, such as about 1 μm or less, such as about 0.75 μm or less, or any ranges or values therebetween.

Additionally or alternatively, the particles can have a D10 particle size distribution value, which is the particle diameter where 10% of the sample's mass includes particles of that size or smaller, of about 3 μm or less, such as about 2.5 μm or less, such as about 2 μm or less, such as about 1.5 μm or less, such as about 1 μm or less, such as about 0.5 μm or less, such as about 0.4 μm or less, such as about 0.3 μm or less, such as about 0.2 μm or less, such as about 0.1 μm or less, such as about 0.05 μm or less, such as about 0.04 μm or less, such as about 0.03 μm or less, such as about 0.01 μm or less, or any ranges or values therebetween.

Nevertheless, in embodiments, the co-particulated milk protein composition may contain greater than or about 50 wt. % whey protein, based on the weight of total true protein, such as greater than or about 52.5 wt. %, such as greater than or about 55 wt. %, such as greater than or about 57.5 wt. %, such as greater than or about 60 wt. %, such as greater than or about 62.5 wt. %, such as greater than or about 65 wt. %, such as greater than or about 67.5 wt. %, such as greater than or about 70 wt. %, such as greater than or about 72.5 wt. %, such as greater than or about 75 wt. %, such as greater than or about 77.5 wt. %, such as greater than or about 80 wt. % such as greater than or about 82.5 wt. %, such as greater than or about 85 wt. %, such as greater than or about 87.5 wt. %, such as greater than or about 90 wt. %, such as greater than or about 92.5 wt. %, such as greater than or about 95 wt. % whey proteins based on the weight of total true protein, or any ranges or values therebetween.

In addition, in embodiments, the co-particulated milk protein composition may contain less than or about 50 wt. % casein protein, based on the weight of total true protein, such as less than or about 47.5 wt. %, such as less than or about 45 wt. %, such as less than or about 42.5 wt. %, such as less than or about 40 wt. %, such as less than or about 37.5 wt. %, such as less than or about 35 wt. %, such as less than or about 32.5 wt. %, such as less than or about 30 wt. %, such as less than or about 27.5 wt. %, such as less than or about 25 wt. %, such as less than or about 22.5 wt. %, such as less than or about 20 wt. %, such as less than or about 17.5 wt. %, such as less than or about 15 wt. %, such as less than or about 12.5 wt. %, such as less than or about 10 wt. % such as less than or about 7.5 wt. %, such as less than or about 5 wt. %, such as greater than or about 2.5 wt. %, such as greater than or about 5 wt. % casein proteins based on the weight of total true protein, or any ranges or values therebetween.

Furthermore, the present technology has found that by utilizing specific ratios of whey proteins to casein proteins, favorable particle sizes and process viscosities may be further tailored, as will be discussed in greater detail in regards to the examples below. Thus, in embodiments, a ratio of whey protein to casein protein in the co-particulated milk protein composition (and/or ratio of the starting whey protein composition and casein source) may be from about 60:40 to about 95:5, such as greater than or about 65:35, such as greater than or about 70:30, such as greater than or about 75:25, such as greater than or about 80:20, such as greater than or about 85:15, such as greater than or about 90:10, up to about 95:5, or any ranges or values therebetween.

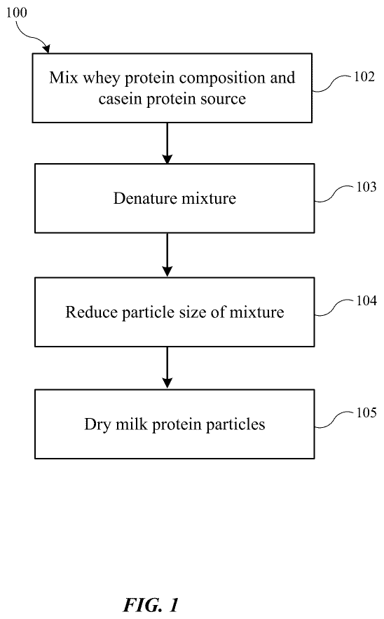

Regardless of the type of proteins present, the co-particulated milk protein composition according to the present technology may be predominantly protein, and may therefore contain greater than or about 60 wt. % total protein on a dry weight basis, based on the weight of the co-particulated milk protein composition, such as greater than or about 62.5 wt. %, such as greater than or about 65 wt. %, such as greater than or about 67.5 wt. %, such as greater than or about 70 wt. %, such as greater than or about 72.5 wt. %, such as greater than or about 75 wt. %, such as greater than or about 77.5 wt. %, such as greater than or about 80 wt. %, such as greater than or about 82.5 wt. %, such as greater than or about 85 wt. %, such as greater than or about 87.5 wt. %, such as greater than or about 90 wt. % such as greater than or about 92.5 wt. %, such as greater than or about 95 wt. % total protein on a dry weight basis, based upon the weight of the co-particulated milk protein composition, or any ranges or values therebetween. The above discussed weight ratios may also be applicable to the whey protein composition and casein protein sources discussed below in regards to FIG. 1 and methods utilized herein.

FIG. 1 shows exemplary operations in a method 100 according to some embodiments of the present technology. The method may be performed in a variety of processing apparatus as known in the art. Method 100 may include a number of optional operations, which may or may not be specifically associated with some embodiments of methods according to the present technology. For example, many of the operations are described to provide a broader scope of the composition formation, but are not critical to the technology, or may be performed by alternative methodology as would be readily appreciated.

The whey composition feedstock utilized herein may be obtained from a cheesemaking process, a whey protein concentrate, a whey protein isolate, native whey protein concentrate, native whey protein isolate, such as one or more whey feedstocks derived from cow's milk, or a combination thereof. In some embodiments, the whey feedstock may be generated from the cheesemaking process and may be referred to as “sweet whey” when the cheesemaking process uses rennet enzymes like chymosin, and “acid whey” when acids are used to form the curds. The pH of sweet whey typically ranges from about 5.6 to 6.6, while the pH of acid whey typically ranges from 4.3 to 4.6. While any appropriate whey feedstock may be utilized, in some embodiments, the whey feedstock utilized may include a whey protein concentrate, whey protein isolate, native whey protein concentrate, native whey protein isolate, acid whey, or a combination thereof.

In some embodiments, the native GMP levels of the whey protein or whey and casein feedstock may be reduced utilizing one or more enzymes that selectively reduce native GMP levels without hydrolyzing the β-lactoglobulin, α-lactalbumin, and/or casein proteins. GMP may also be reduced through the use of chromatography systems, or by using native whey protein isolate purified directly from milk that has not undergone cheesemaking or acid whey, which naturally contains little to no native GMP, or blending such native whey protein isolate or acid whey with whey protein concentrates from cheese-making (e.g. sweet whey). In embodiments, the one or more enzymes can be one or more protease enzymes. Sources for the one or more protease enzymes may include microorganisms, fungi, plant, and/or animal sources, among others. For example, the one or more protease enzymes may be derived from fungi of the genus Aspergillus, bacteria of the genus Bacillus (e.g., Bacillus subtilis), and/or animals (e.g., trypsin, chymotrypsin, etc.), among other sources. Nonetheless, in some embodiments, the one or more protease enzymes include an acid protease enzyme, a neutral protease enzyme, an alkaline protease enzyme, or a combination thereof. However, in some embodiments, the one or more protease enzymes include a neutral protease enzyme, an alkaline protease enzyme, or a combination thereof. Additionally, or alternatively, the protease enzyme can be an endoprotease, an exoprotease, or a combination thereof. Thus, in some embodiments, the one or more protease enzymes can include an aspartic protease, serine protease, a cysteine protease, or a combination thereof. Nonetheless, in one embodiment, the one or more protease enzymes can include a serine protease, such as an alkaline serine protease, alone or in combination with at least one neutral protease enzyme.

Notwithstanding the one or more enzymes selected, the one or more enzymes selected may be added to the whey protein and/or milk protein feedstock in an amount of about 0.001 wt. % or greater, based upon the weight of the total protein in the composition, such as about 0.0025 wt. % or greater, such as about 0.005 wt. % or greater, such as about 0.0075 wt. % or greater, such as about 0.01 wt. % or greater, or any ranges or values therebetween. It should be understood that the foregoing ranges may refer to a total amount of enzymes included in the whey protein feedstock, or to an amount of each enzyme added to the whey protein feedstock.

However, in one aspect, the amount of the one or more enzymes added is selected so as to hydrolyze at least about 10 wt. % or more of the native GMP present in the whey protein feedstock, such as about 15 wt. % or more, such as about 20 wt. % or more, such as about 25 wt. % or more, such as about 30 wt. % or more, such as about 35 wt. % or more, such as about 40 wt. % or more, such as about 45 wt. % or more, such as about 50 wt. % or more, such as about 60 wt. % or more, such as about 65 wt. % or more, such as about 70 wt. % or more, such as about 72.5 wt. % or more, such as about 75 wt. % or more, such as about 80 wt. % or more, such as about 85 wt. % or more, or any ranges or values therebetween. Stated differently, in some embodiments, the co-particulated milk protein composition according to the present technology may have a reduced amount of native GMP as compared to a whey protein feedstock, according to any one or more of the above percentages.

Regardless of the amount of the one or more enzymes selected, the GMP selective enzyme(s) are added to the whey protein feedstock and/or mixed whey-casein feedstock to hydrolyze the native GMP in the co-particulated milk protein composition. In some embodiments, the hydrolysis phase can continue for about 72 hours or less, such as about 60 hours or less, such as about 48 hours or less, such as about 36 hours or less, such as about 24 hours or less, such as about 12 hours or less, such as about 10 hours or less, such as about 8 hours or less, such as about 5 hours or more, or any ranges or values therebetween. The hydrolysis may occur at a temperature of about 60° F. or less, such as about 55° F. or less, such as about 50° F. or less, such as about 45° F. or less, or any ranges or values therebetween.

In some embodiments, the low native GMP and high fat levels discussed above in the high fat feedstock can be obtained or improved by utilizing a microfiltration membrane (such as a microfiltration membrane having a pore size of about 0.5 micrometers or less, such as about 0.4 micrometers or less, such as about 0.3 micrometers or less, or such as about 0.08 micrometers or greater, or any ranges therebetween) filtration process. Namely, in some embodiments, a microfiltration membrane process can be selected so as to retain denatured whey proteins and fat while allowing some or all native proteins, including native GMP to pass through to a permeate side. It should be understood that, in some embodiments, other filtration methods may be utilized to provide the high fat, low native GMP feedstock.

However, in embodiments where a microfiltration membrane was utilized, it was also surprisingly found that the ratio of β-lactoglobulin to α-lactalbumin can be increased. Namely, without wishing to be bound by theory, β-lactoglobulin may be more prone to early denaturation compared to α-lactalbumin. Thus, the β-lactoglobulin may largely be retained by the membrane with the denatured whey proteins whereas a higher proportion of the α-lactalbumin passes through with the permeate. In some embodiments, a whey protein feedstock composition according to the present technology exhibits a ratio of β-lactoglobulin to α-lactalbumin of about 2.75 or greater, such as about 3 or greater, such as about 3.5 or greater, such as about 4 or greater, such as about 4.5 or greater, such as about 5 or greater, such as about 5.5 or greater, such as about 6 or greater, such as about 6.5 or greater, such as about 7 or greater, such as about 7.5 or greater, or any ranges or values therebetween.

For instance, in some embodiments, a whey protein feedstock composition can include about 65 wt. % β-lactoglobulin or more, such as about 67.5 wt. % or greater, such as about 70 wt. % or greater, such as about 72.5 wt. % or greater, such as about 75 wt. % or greater, or any ranges or values therebetween. Additionally, or alternatively, in embodiments, a whey protein feedstock composition can include about 15 wt. % or less α-lactalbumin, such as about 12.5 wt. % or less, such as about 10 wt. % or less, such as about 7.5 wt. % or less, or such as greater than or about 7 wt. %, or any ranges or values therebetween. Nonetheless, it should be understood that, in embodiments, no reduction in GMP may be necessary.

Notwithstanding the whey protein composition feedstock selected, at operation 102, the whey protein composition may be mixed with a casein protein source. Suitable casein protein sources (also referred to as feedstock herein), may include micellar casein concentrate, micellar casein isolate, ultrafiltered milk protein, microfiltered milk protein, milk protein concentrate, a caseinate, rennet casein, acid casein, or a combination thereof. Regardless of the casein protein source selected, the casein protein source should contain one or more casein proteins having a cysteine containing-residue, as discussed above.

While any one or more of the feedstocks for the whey protein and casein protein may be utilized, the mixture of casein protein source and whey protein composition may be subjected to denaturation 103 as known in the art. For instance, in some embodiments, the mixture of milk protein particles may be denatured by being heated to a temperature greater than or about 70° C., such as greater than or about 72.5° C., such as greater than or about 75° C., such as greater than or about 77.5° C., such as greater than or about 80° C., such as greater than or about 82.5° C., such as greater than or about 85° C., such as greater than or about 87.5° C., such as greater than or about 90° C., such as greater than or about 92.5° C., such as greater than or about 95° C., such as greater than or about 97.5° C., such as greater than or about 100° C., or any ranges or values therebetween, where the heating converts at least a portion of the starting milk proteins to denatured milk proteins. Advantageously, such heating also denatures any enzymes, if utilized. Concurrent with the heating, the slurry may be mixed or agitated to reduce the level of aggregation of the denaturing proteins at operation. The slurry may be mixed and heated for about 1 second to about 120 seconds, such as about 2.5 seconds to about 105 seconds, such as about 5 seconds to about 90 seconds, or any ranges or values therebetween.

Namely, it was found that the distribution of whey proteins between forming aggregates and encasing the casein micelles remains relatively stable when subjected to heat treatments within the range of 70° C. to 100° C. However, modifying the pH to fall between 6 and 7 before applying heat treatment influences the denaturation process. The overall extent of denaturation remains fairly consistent, but employing higher pH levels during heat treatment leads to an increased formation of whey protein aggregates, whereas lower pH levels during heat treatment foster a stronger association between whey proteins and the casein micelles. Thus, in embodiments, the denaturing operation may occur at a pH of less than or about 7, such as less than or about 6.9, such as less than or about 6.8, such as less than or about 6.7, such as less than or about 6.6, such as less than or about 6.5, such as less than or about 6.4, or such as greater than or about 6, such as greater than or about 6.1, such as greater than or about 6.2, such as greater than or about 6.3, such as greater than or about 6.4, or any ranges or values therebetween.

During or after heating, the denatured milk protein composition can be subjected to mechanical shear conditions 104 to reduce a particle size of the co-particulated milk protein particles in the denatured mixture. In some embodiments, mechanical shear conditions may further denature the milk proteins and/or deactivate the one or more enzymes or may reduce the aggregates that may have formed as the proteins denature. Mechanical shear conditions as used herein generally refer to high shear conditions in which at least about 1,000 s⁻¹ of shear is applied, such as greater than or about 10,000 s⁻¹ of shear is applied, such as greater than or about 50,000 s⁻¹ of shear is applied, such as greater than or about 100,000 s⁻¹ of shear is applied, up to about 500,000 s⁻¹ of shear is applied. In some embodiments, the denatured milk protein composition is typically sheared by a high-shear mixer, colloid mill, or swept surface heat exchanger at a temperature of about 120 to 300° F. for about 0.1 to 120 seconds.

Nonetheless, after heating, the co-particulated milk protein composition of the present technology, the final protein contains about 45 wt. % or more denatured milk proteins relative to the total weight of the protein in the co-particulated milk protein composition, such as about 50 wt. % or more, such as about 55 wt. % or more, such as about 60 wt. % or more, such as about 65 wt. % or more, such as about 70 wt. % or more, such as about 75 wt. % or more, such as about 77.5 wt. % or more, such as about 80 wt. % or more, such as about 85 wt. % or more, such as about 90 wt. % or more, or any ranges or values therebetween.

Notwithstanding the final composition of proteins, the co-particulated milk protein composition may be optionally cooled and concentrated after heating and shearing, following by drying 105 to produce a powdered co-particulated milk protein composition. Drying processes may include spray drying, heating, and evaporation, among other processes. As will be discussed in greater detail below, the co-particulated milk protein composition may then be packaged or added directly to other ingredients for making a food or beverage composition.

Regardless of whether GMP reduction was conducted, a co-particulated milk protein composition according to the present technology can include less than 12 wt. % native GMP based upon the total weight of protein in the co-particulated milk protein composition, such as about 11 wt. % or less, such as about 10 wt. % or less, such as about 9 wt. % or less, such as about 8 wt. % or less, such as about 7 wt. % or less, such as about 6.5 wt. % or less, such as about 6 wt. % or less, such as about 5.9 wt. % or less, such as about 5 wt. % or less, such as about 4 wt. % or less, such as about 3 wt. % or less, such as about 2 wt. % or less, such as about 1 wt. % or less, such as about 0.5 wt. % or less, such as about 0.1 wt. % or less, such as about 0.05 wt. % or less, such as about 0 wt. %, or any ranges or values therebetween.

Thus, in some embodiments, the co-particulated milk protein composition may exhibit a weight ratio of native GMP to co-particulated milk protein of about 0.15 or less, such as about 0.125 or less, such as about 0.1 or less, such as about 0.09 or less, such as about 0.085 or less, such as 0.0, or any ranges or values therebetween.

In addition, as may be understood from the above, during hydrolysis of the GMP, hydrolyzed GMP is formed, and can advantageously remain in the co-particulated milk protein composition based upon the desired end use. Namely, the present technology has found that the denatured GMP (GMP that has undergone at least one hydrolysis reaction, also referred to herein as enzymatically hydrolyzed GMP) does not exhibit the same adverse effects as the native GMP (such as cardboard flavor/artificial flavor). Thus, in some embodiments, the co-particulated milk protein composition can contain about 1 wt. % or more of denatured GMP based upon the total weight of protein in the co-particulated milk protein composition, such as about 2 wt. % or more, such as about 3 wt. % or more, such as about 4 wt. % or more, such as about 5 wt. % or more, such as about 6 wt. % or more, such as about 7 wt. % or more, such as about 8 wt. % or more, such as about 9 wt. % or more, or any ranges or values therebetween. However, in embodiments, little to no hydrolyzed GMP may remain in the co-particulated milk protein composition, and in some embodiments, less than 1 wt. % native or hydrolyzed GMP, based on a weight of true protein, may be contained in the co-particulated milk protein composition.

Similarly, as the native GMP is acted upon by one or more enzymes, the co-particulated milk protein composition may have an increased proteolysis index in some embodiments. Proteolysis index is a measure of the increase of non-protein nitrogen (NPN) in relation to the total Kjeldahl nitrogen (TKN) of a sample, for which the method for determining is described in the examples below. Namely, the proteolysis index (PI) increases in a sample as protein is broken down by enzymatic activity to its primary amino acids or small peptides that become soluble in trichloroacetic acid, known as non-protein nitrogen, and can therefore be an indicator for protein hydrolysates. Thus, in some embodiments, the co-particulated milk protein composition according to the present technology may have a proteolysis index of about 6 wt. % or greater, such as about 7 wt. % or greater, such as about 8 wt. % or greater, such as about 9 wt. % or greater, such as about 10 wt. % or greater, such as about 12.5 wt. % or greater, such as about 15 wt. % or greater, such as about 17.5 wt. % or greater, such as about 20 wt. % or greater, such as about 22.5 wt. % or greater, such as about 25 wt. % or greater, or any ranges or values therebetween.

Furthermore, in embodiments, the co-particulated milk protein composition contains less than 10 wt. % of fat on a dry weight basis, based on the weight of the co-particulated milk protein composition, such as less than or about 9 wt. %, such as less than or about 8 wt. %, such as less than or about 7 wt. %, or any ranges or values therebetween.

As discussed above, in embodiments, the co-particulated milk protein composition may have a carefully controlled α-lactalbumin level. Thus, in embodiments, the co-particulated milk protein composition may contain about 12.5 wt. % or less, such as about 10 wt. % or less, such as about 7.5 wt. % or less, or such as greater than or about 7 wt. %, such as greater than or about 8 wt. %, such as greater than or about 9 wt. %, such as greater than or about 10 wt. % α-lactalbumin based on the weight of true protein, or any ranges or values therebetween.

As discussed above, the co-particulated milk protein composition can be packed in powdered form or can be incorporated into a food or beverage product to produce a fortified food and/or beverage. Suitable food and beverage products can include protein bars, granola bars, yogurts, drinkable yogurts, pudding products, ready to drink beverages, ready to mix beverage powders, bakery products, medical nutrition products, nutraceutical products, meat products, cheese, butter, grain products, cream cheese, milk products, and the like. Fortified foods and or beverages as discussed herein may contain one or more excipients as known in the arts. Exemplary excipients may include flavorings, such as fruit, vegetables, fruit flavorings, and/or vegetable flavorings, preservatives, sweeteners, savory flavorings, lactose, cream, and the like, as well as combinations thereof.

As an example only of a product and a method for fortifying the product utilizing a co-particulated milk protein composition as discussed herein, a yogurt composition may be formed as known in the art. For instance, a spoonable or drinkable yogurt milk, which may contain whole milk, defatted milk, or a combination thereof fortified with a co-particulated milk protein composition according to the present technology, may be sent to a fermenter, where yogurt cultures are added, yielding a yogurt mix. The yogurt mix may then be further concentrated and combined with further ingredients or flavorings and packaged.

A fortified food and/or beverage product as discussed herein may have a viscosity, the method for which will be discussed in greater detail in the examples, of about 500 centipoise (cP) or less, such as about 400 centipoise or less, such as about 300 centipoise or less, such as about 250 centipoise or less, such as about 200 centipoise or less, such as about 150 centipoise or less, such as about 100 centipoise or less, such as about 50 centipoise or less, or any ranges or values therebetween.

For instance, in embodiments, drinkable yoghurts fortified with co-particulated milk protein compositions according to the present technology may exhibit a viscosity of less than or about 300 centipoise or less, such as about 250 centipoise or less, such as about 200 centipoise or less, such as about 150 centipoise or less, such as about 125 centipoise or less, such as about 100 centipoise or less, such as about 90 centipoise or less, such as about 80 centipoise or less, such as about 70 centipoise or less, such as about 60 centipoise or less, such as about 50 centipoise or less, such as about 40 centipoise or less, such as about 30 centipoise or less, such as about 25 centipoise or less, or such as about 10 centipoise or greater, or any ranges or values therebetween, at fortification levels of 10 wt. % total protein in the fortified food and/or beverage.

For instance, in embodiments, spoonable yoghurts fortified with co-particulated milk protein compositions according to the present technology may exhibit a viscosity of about 5,000 centipoise to about 22,000 centipoise, such as about 22,500 centipoise or less, such as about 22,000 centipoise or less, such as about 21,000 centipoise or less, such as about 20,000 centipoise or less, such as about 19,000 centipoise or less, such as about 18,500 centipoise or less, or such as greater than or about 7,500 centipoise, such as greater than or about 10,000 centipoise, such as greater than or about 12,500 centipoise, such as greater than or about 15,000 centipoise, such as greater than or about 17,500 centipoise, or any ranges or values therebetween, at fortification levels of 7 wt. % total protein in the fortified food and/or beverage.

Furthermore, a fortified food and/or beverage product as discussed herein can be fortified to a protein level of greater than 3%, or about 8 wt. % or greater, based upon the weight of the food and/or beverage product, such as about 8.5 wt. % or greater, such as about 9 wt. % or greater, such as about 9.5 wt. % or greater, such as about 10 wt. % or greater, such as about 12.5 wt. % or greater, such as about 15 wt. % or greater, such as about 17.5 wt. % or greater, such as about 20 wt. % or greater, such as about 22.5 wt. % or greater, such as about 25 wt. % or greater, such as about 27.5 wt. % or greater, such as about 30 wt. % or greater, or any ranges or values therebetween.

Furthermore, certain embodiments of the present disclosure may be better understood according to the following examples, which are intended to be non-limiting and exemplary in nature.

### EXAMPLES

### Test Methods and Procedures

### Quantification of Glycomacropeptide (GMP), Alpha-lactalbumin (Alpha-La) and Beta-lactoglobulin (Beta-Lg)

Quantification of GMP was determined by Beckman Capillary Electrophoresis system. The capillary is a DOV-1701OH Deactivated TSP standard FS tubing (600 mm×50 μm) with a slit opening of 100×800 μm and regenerated/activated with 0.1N HCl solution.

### Preparation of Sample Solution

Prepare a 1% Protein solution with deionized (DI) water of each sample
Vortex samples until homogenous.
Let powder samples hydrate for at least 30 minutes before continuing with sample prep.
Prepare sample buffer using 0.0787 g of DTT (threo-1,4-Dimercapto-2,3-butanediol) and 30 g of reducing buffer. Reducing buffer consists of 167 mM Tris, 42 mM 3-morpholino-propanesulfonic acid, 67 mM ethylenedinitrilotetraacetic acid disodium, 0.5 g/L methyl hydroxypropyl cellulose in 8M urea solution.
After samples are hydrated, mix sample buffer and sample in a 1:1 ratio to a total of 4 mL.
Vortex the sample and let sit for one hour.

Filter samples into glass vials using PVDF 0.22 μm syringe filter. Put caps on glass vials. Sample solution is injected for 10 s at 3.4 Kpa. The separation is conducted under 45° C. at 25 KV (increasing from 0 to 25 KV within 3 min initially). Detection of milk proteins is performed at 214 nm. Quantity of each protein component is determined by comparing the peak area of each component peak to the total peak areas. On this example:

$$GMP \% = \frac{Peak area for GMP}{Sum of Peak areas for all proteins} \times 100 \%$$

$$\frac{Beta\_Lg}{Alpha\_La} = \frac{Peak area for Beta\_Lg}{Peak area for Alpha\_La}$$

### Quantification of Fat

The fat component is determined by the Mojonnier modification of the Roese-Gottlieb procedure for fat extraction (Reference method AOAC 989.05). The Roese-Gottlieb procedure uses ether to extract the fat from dairy products.

### Calculate:

$$Fat \% = \frac{Weight of Fat - Blank}{weight of sample} \times 100 \%$$

### Where:

Weight of Fat: (weight sample beaker after extraction)−(the weight of the empty beaker).
Blank: an analytical blank determination must be subtracted from the weight of fat obtained.
Weight of Sample: weight of sample prior to extraction procedure.

### Quantification of Protein Analysis by Total Kjeldahl Nitrogen (TKN)

The protein analysis by Kjeldahl Nitrogen (Reference method AOAC 991.20) is used to determine the percent of nitrogenous compounds by weight. Sulfuric acid digests proteins and other nitrogenous compounds converting the nitrogen into ammonium sulfate. A catalyst is used to increase the reaction rate and to raise the boiling point of sulfuric acid. Titration of the ammonia with standard hydrochloric acid gives the amount of nitrogen associated with protein and soluble nitrogen.

### Determination of TKN (Crude Total Protein)

$$T K N \% = \frac{Δ mL * N * 14.007 * 6.38}{10 * W}$$

### Where:

ΔmL=the amount of standardized HCl added to the sample−the amount added to the blank in mL (often calculated by the distillation apparatus and given as ΔmL).
N=the exact normality of the standardized HCl from the Certificate of Analysis in meq/mL units.
14.007=Formula weight of nitrogen in mg/meq.
The constant 6.38 is the number of grams of dairy protein per gram of nitrogen. Other constants are 4.7218 for ammonium sulfate, and 7.2904 for 1-tryptophan.
W=the sample size in grams.
Division by 10 gives the result as g per 100 grams.
The result is expressed as the percentage of protein by weight (#of grams per 100 grams sample).

### Determination of NPN (Non-Protein Nitrogen)

Non-protein nitrogen (NPN) consists of urea, ammonia, free amino acids, creatine, uric acid, peptides, and amino alcohols of phospholipids (Ruska and Jonkus 2014) which are soluble in trichloroacetic acid. Generally greater than 30 amino acids are sufficient to qualify a polypeptide as protein, though not a definitive rule, peptide consisting of less than 30 amino acids likely are found in the TCA soluble fraction and classified as non-protein nitrogen. The method uses addition of trichloroacetic acid (TCA) to precipitate proteins. The protein is filtered out and the non-protein nitrogen determined in the filtrate. The amount of non-protein nitrogen can then be determined.

### Sample Preparation

A. Mix sample well.

B. Tare a 150 ml beaker or 4 oz snap-cap. For powder or solid samples, add 10-15 glass balls to the beaker or snap cap before tare.

C. Transfer appropriate number of samples to the tared beaker base on the table below. Record sample weight to nearest 0.0001 g. For powder or solid samples, add 20 mL of DI water and shake to constitute the sample.

D. Using a graduated cylinder or automatic dispenser, add 15 mL of 33% TCA to the sample.

E. Place the beaker or snap-cap back on the balance.

F. Add DI water to the sample until the total mass of sample, TCA solution, and added water, is approximately 50 g. Record gross weight.

G. Mix well, let solution stand for 10 minutes.

H. Filter through slow filter paper, into a clean snap cap or beaker.

I. Transfer approximately 7-10 g filtrate to a Kjeldahl digestion tube. Record the exact mass of filtrate added.

J. Test on TKN (Total Kjeldahl Nitrogen) method to determine Nitrogen in the filtrate (N) for the calculation below:

$$N P N \% = \frac{mL * N * 14.007 * 6.38}{W * A / B / 10}$$

mL=the amount of standardized HCl required to titrate the sample distillate.
N=the exact normality (meq/mL) of the standardized HCl from the lot Certificate of Analysis (COA) or as determined in the laboratory.
14.007=Formula weight of nitrogen in mg/meq.
6.38 is factor converting nitrogen to dairy protein
W=The grams of sample filtrate used
A=the grams of sample used
B=the total grams of sample solution
10 convert the result to grams per 100 grams.

### Determination of True Protein

True protein is the measure of total nitrogen (TKN) (sometimes referred to as crude protein), with the non-protein nitrogen (NPN) content subtracted out. True Protein=TKN-NPN and is expressed as a percentage of true protein by weight (grams of true protein per 100 grams of sample).

### Determination of Proteolysis Index (PI)

Proteolysis index is a measure of the increase of non-protein nitrogen (NPN) in relation to the total Kjeldahl nitrogen (TKN) of a sample. The proteolysis index (PI) increases in a sample as protein is broken down by enzymatic activity to its primary amino acids or small peptides that become soluble in trichloroacetic acid, known as non-protein nitrogen.

$$P I = \frac{N P N \%}{T K N \%} \times 100 \%$$

Proteolysis index is expressed as NPN percentage of total crude protein (TKN) by weight (#of grams of NPN per 100 grams of Total crude protein).

### Determination of Denatured Protein

The denatured protein method is a measure of proteins that have undergone a disruption and possible destruction of both secondary and tertiary structures rendering the protein insoluble in an environment in which the native protein would typically be soluble.

### Sample Preparation

Prepare a solution of sample at approximately 1.2% (w/w) protein. Adjust pH to 6.8 using either 0.1N HCl or 0.1N NaOH.
Record sample weight and final solution weight after pH adjustment.
Split volume into two fractions for testing—
   10 ml for Total TKN. Test for TKN. This will be % Total TKN.
   25 g for DWP fractions in a beaker for treatment as follows.

### Determination of Denatured Whey Protein (pH 4.6) Fraction

Add ˜10 ml distilled water to the beaker containing the DWP fraction.
Place small magnetic stir bar into beaker. Place beaker on stir plate, insert pH probe and temperature compensator into the solution. Start stirrer.
Adjust the pH to 4.60±0.02 by adding 1 normal HCl for gross adjustment and 0.1 normal HCl for fine adjustment.
Place the beaker on the balance. Bring weight of solution to ˜50 g (e.g., tare weight plus 50 g) with distilled water. Record weight to nearest 0.0001 g.
Let solution rest at room temperature for one hour. Swirl the beaker contents for 10-15 seconds to mix. Transfer contents to a centrifuge tube.
Place tubes in centrifuge, ensuring that tubes are counterbalanced. Centrifuge at 10° C. at 10,000 rpm for 15 minutes. Do not use brake to speed deceleration.
Without transferring any of the pellet material, transfer ˜ 10 mL of the supernatant liquid to an identified culture tube. This is the undenatured whey protein fraction. Test this supernatant for TKN. This is the “% TKN in the undenatured whey protein fraction”.

### Calculate Denatured Whey Protein (DWP):

$$D W P = 1 - ( \frac{\begin{matrix}T K N \% in undenatured whey protein fraction * \\ ( \frac{final weight of dilution}{Weight of solution diluted} )\end{matrix}}{T K N \%} )$$

### Determination of Particle Size Distribution

Particle size distribution analysis was determined using a Malvern Mastersizer 3000 with Hydro EV. The method parameters were 1.46 Particle refractive index, 0.0001 particle absorption index and 1.33 dispersant refractive index and water as dispersant. The Mie scattering model was used for analysis.

### Analysis of Kappa-casein by Capillary Electrophoresis (CE)

Analysis of kappa-casein was determined by Beckman Capillary Electrophoresis system. The capillary is a DOV-1701OH Deactivated TSP standard FS tubing (600 mm×50 μm) with a slit opening of 100×800 μm and regenerated/activated with 0.1N HCl solution.

### Preparation of Sample Solution:

Prepare a 1% protein solution with DI water of each sample
Vortex samples until homogenous.
Let samples hydrate for at least 30 minutes before continuing with sample prep.
Prepare sample buffer using 0.0787 g of DTT (threo-1,4-Dimercapto-2,3-butanediol) and 30 g of reducing buffer. Reducing buffer consists of 167 mM Tris, 42 mM 3-morpholino-propanesulfonic acid, 67 mM ethylenedinitrilotetraacetic acid disodium, 0.5 g/L methyl hydroxypropyl cellulose in 8M urea solution.
After samples are hydrated, mix sample buffer and sample in a 1:1 ratio to a total of 4 mL.
Vortex the sample and let sit for one hour.
Filter samples into glass vials using PVDF 0.22 μm syringe filter. Put caps on glass vials.

Sample solution is injected for 10 s at 3.4 Kpa. The separation is conducted under 45° C. at 25 KV (increasing from 0 to 25 KV within 3 min initially). Detection of kappa-casein and other milk proteins is performed at 214 nm. Kappa-casein level is determined by comparing the peak area of kappa-casein peak to the total peak areas for all proteins.

$$Kappa Casein \% = \frac{Peak area for Kappa casein}{Sum of Peak areas for all proteins} \times 100 \%$$

### Example 1

### Methods for Forming Co-particulated Milk Protein Compositions

Feed material of co-particulated milk protein composition includes whey protein concentrate (WP), ultrafiltered milk concentrate (UF) or micellar casein isolate (MCI), or condensed skim milk, or any combination of these. Prior to blending or following blending the protein containing ingredients are processed to raise the protein to greater than 60 wt. % protein on a dry weight basis. The co-particulated milk protein composition feed was obtained by forming a mixture on a true protein basis of whey protein and casein sources at a ratio of 90 wt. % whey protein to 10 wt. % casein, with casein sources from the ultrafiltered milk concentrate or micellar casein isolate, followed by adjusting to 14 wt. % protein (operating range 13-33%) with addition of water. The co-particulated milk protein composition feed was held in a tank with agitation at about 45° F. or less before further processing.

Protease enzymes were optionally added to selectively hydrolyze glycomacropeptide (GMP) in the co-particulated milk protein feed. Addition of the protease enzymes start the cold incubation phase of the protein hydrolysis when utilized. In this experiment, the protease enzyme included at least one alkaline serine protease enzyme and at least one neutral protease enzyme. The one or more protease enzymes were added to the cold co-particulated milk protein mixture at a level of about 0.012% by wt. of the total protein in the substrate. For the 5,000 lb. batch with 14 wt. % protein, about 38 g of enzyme was added. The cold incubation phase lasts for at least 5 hours with agitation at ≤45° F.

Denaturation of the co-particulated proteins, optionally treated with protease enzymes, was achieved by preheating the feed to 130° F. (operating range 120-150° F.), then heating to a denaturation temperature of 176-203° F., holding for 1-120 sec, and mechanically shearing during the heating for particle size control. Following the heating and shear process, the denatured whey protein composition was cooled to a temperature below 45° F., followed by spray drying to produce a powdered co-particulated milk protein composition.

### Example 2

### Co-particulated Milk Protein Particle Size Analysis

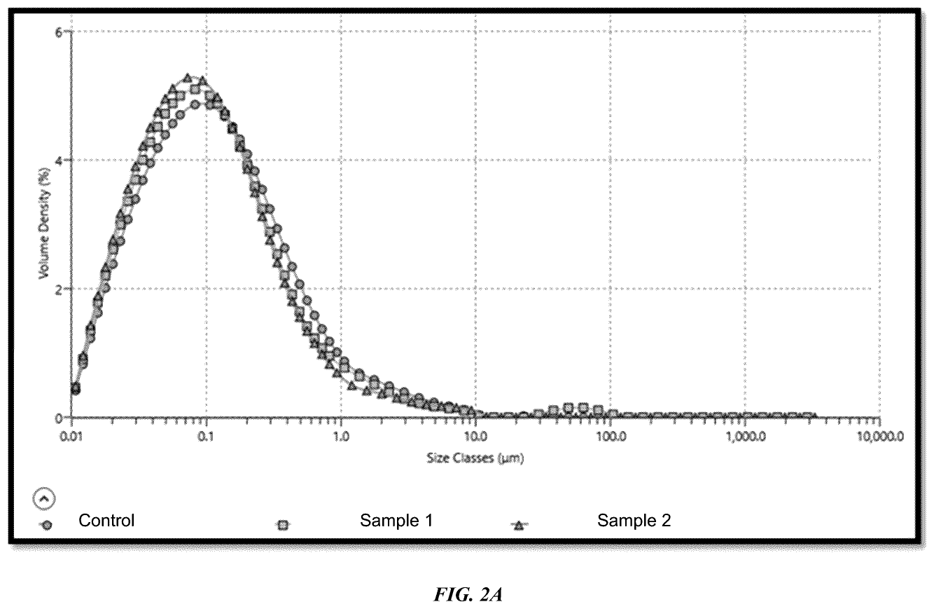

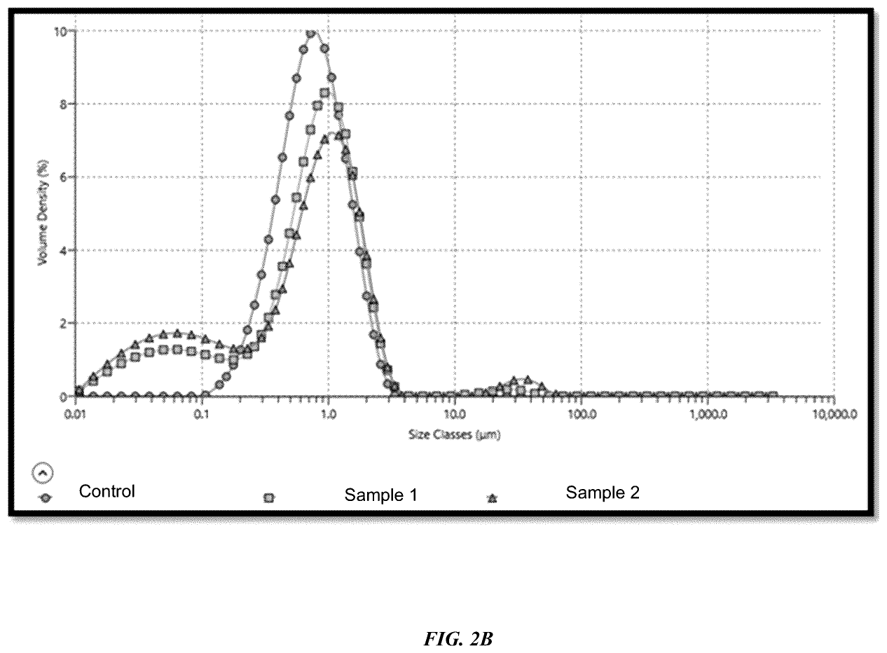

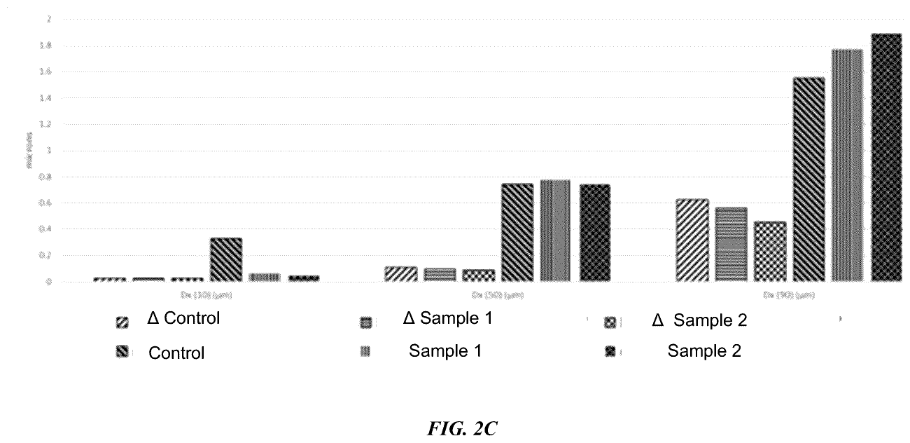

Solutions of: whey protein concentrate (WP), ultrafiltered milk concentrate (UF) and micellar casein isolate (MCI) each with a protein content of at least 80% protein on a dry weight basis were collected. Particle size of the whey protein which is labelled “Control”, 90% whey protein to 10% casein (true protein basis) with casein from ultrafiltered milk which is labeled “Sample 1”, and 90% whey protein to 10% casein (true protein basis) with casein from micellar casein isolate which is labelled “Sample 2” were measured as shown in FIG. 2A and Table 1. Particle size analysis demonstrates no significant difference as 90% of all particles are less than 0.70 microns.

TABLE 1

| Sample Name | Dx (10) (μm) | Dx (50) (μm) | Dx (90) (μm) |
|---|---|---|---|
| Control | 0.0252 | 0.106 | 0.622 |
| Sample 1 | 0.0241 | 0.0945 | 0.558 |
| Sample 2 | 0.0234 | 0.0879 | 0.453 |

Following thermal treatment as outlined in Example 1 particle size was again measured as illustrated in FIG. 2B and Table 2. Following thermal treatment, the overall particle size of the whey protein sample (Control) increased significantly following thermal denaturation. For instance, as illustrated in FIG. 2C, thermal treatment of the control sample (whey protein only) exhibited a D10 increase from 0.025 (untreated) to 0.33 microns as shown by the reference referred to as Δ control, whereas sample 1 and sample 2 exhibited only a slight increase in D10 particle size, as illustrated by references Δ sample 1 and Δ sample 2, which reflect the change in particle size from untreated (FIG. 2A) to treated (FIG. 2B). For D50 particle size of all products observed approximately the same increase in size. The D90 particles of sample 1 and sample 2 created an inverse relationship to that of the feed materials (untreated, FIG. 2A) where the control had the largest untreated D90 particle size, the resulting thermal treatment caused the control to exhibit the smallest treated D90 particle size. This suggests that the presence of casein during co-particulation of whey and casein blends results in larger particles as whey and casein covalently interact during thermal treatment.

TABLE 2

| Sample Name | Dx (10) (μm) | Dx (50) (μm) | Dx (90) (μm) |
|---|---|---|---|
| Control | 0.327 | 0.741 | 1.55 |
| Sample 1 | 0.0602 | 0.775 | 1.77 |
| Sample 2 | 0.0449 | 0.74 | 1.89 |

### Example 3

### Co-Particulated Milk Protein: Evaluation of Aggregated Protein Particles

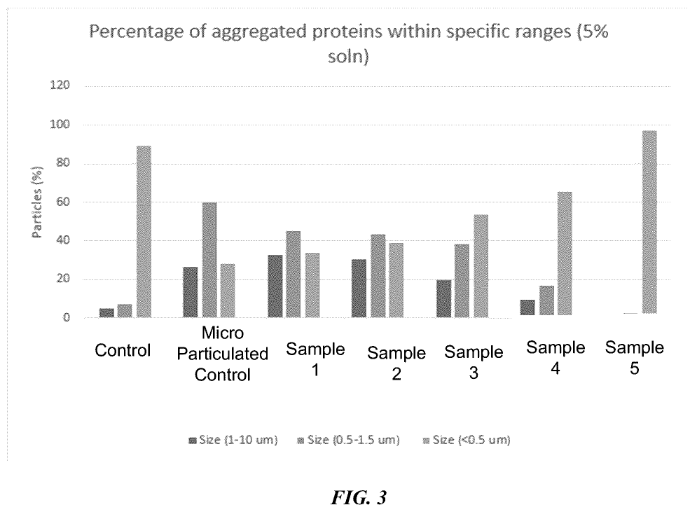

Co-particulated milk proteins containing from 10 wt. % to 75 wt. % % casein was evaluated for particles size using laser diffraction in a 5% solution of particulated proteins that had hydrated for 1-hour and then homogenized at 100 bars. The particles size expressed in FIG. 3A is the percentage of particles present between 1-10 microns between 0.5-1.5 microns, and less than 0.5 microns. Co-particulated proteins are compared to a microparticulated whey protein control (“microparticulated control”) and non-microparticulated WP80 (“Control” 80 wt. % whey protein concentrate, on a dry matter basis). Samples 3-5 were formed in the same manner as Example 1, and contain a blend of 70% whey protein to 30% casein (true protein basis) with casein from micellar casein isolate (Sample 3), a blend of 50% whey protein to 50% casein (true protein basis) with casein from micellar casein isolate (Sample 4), and a blend of 25% whey protein to 75% casein (true protein basis) with casein from micellar casein isolate (Sample 5).

While the percentage of particles within 0.5-1.5 microns decrease steadily as casein content of co-particulated milk proteins increases, the particles that are less than 0.5 microns steadily increase suggesting that as casein increases there is more casein that is not interacting with whey proteins during thermal treatment.

### Example 4

### Yogurt Fermentation Time

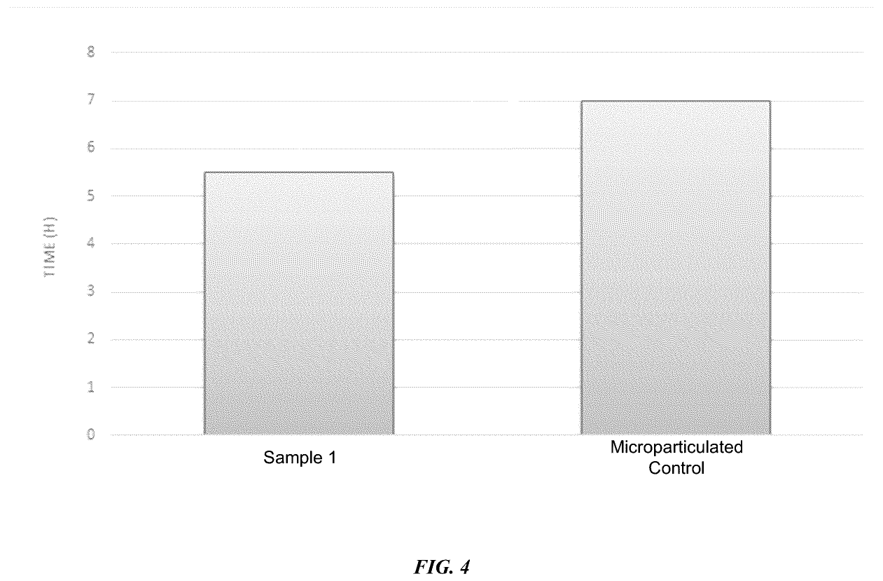

FIG. 4A illustrates fermentation time of co-particulated milk proteins relative to microparticulated whey protein. FIG. 4 shows the total fermentation times of yogurts prepared with different particulated proteins when fermented with 0.02% by weight of starter culture at 104° F. The fermentation time is the time required for the pH to reach 4.6. Data provided in this example shows that yogurt made with co-particulated milk proteins (sample 1) requires significantly less time to reach pH 4.6 when compared to yogurt using microparticulated whey protein (microparticulated control). This demonstrates that the fermentation time can be reduced by 14 to 28% when utilizing co-particulated milk protein compositions discussed herein. This reduction in time is a significant improvement on production efficiency and waste. Similarly, this finding could help optimize the starter culture usage and reduce waste and excess starter and process materials.

### Example 5

### Drinkable Yogurt

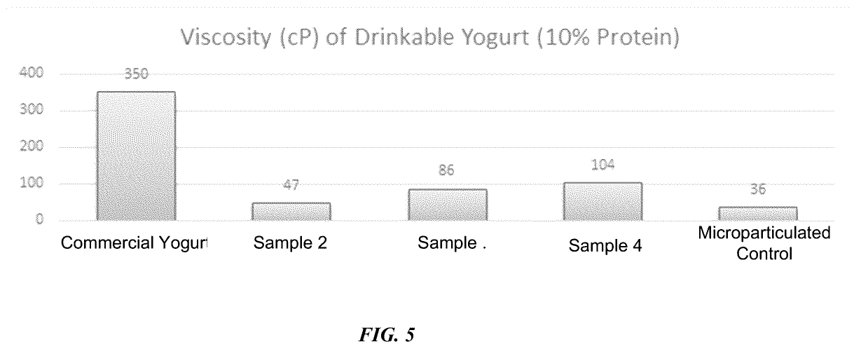

Various levels of protein fortification may be of interest to consumers. Examples of protein fortification in drinkable yogurt is illustrated in Table 3 and FIG. 5. Samples 3, 4, and 5 were formed in the same manner as Example 1. As discussed above, Sample 2 contains a 90:10 blend of whey protein to micellar casein isolate, Sample 3 contains a 70:30 blend of whey protein to micellar casein isolate, and Sample 4 contains a 50:50 blend of whey protein to micellar casein isolate. As illustrated, a drinkable yogurt fortified to 10 wt. % protein with co-particulated milk protein compositions according to the present technology, have a preferred lower viscosity when compared to a commercially available product having a lower 6.7% protein level. Thus, the present technology exhibits a marked decrease in viscosity even at higher fortification levels. The reduced viscosity is beneficial for the consumer experience due to increasing protein per serving and ease of consuming the product.

TABLE 3

| | Drinkable Yogurt base Recipes | |
|---|---|---|
| | 10% Protein after fortification | |
| | w/mpWP or cpMilk-protein | |
| | W/W % in | W/W % range in |
| Ingredients | base recipe | base recipe |
| Skim milk | 90.8 | 90.6-91.0 |
| Ultrafiltered Milk | 0 | 0 |
| Microparticulated control or Co- | 9.2 | 9.0-9.4 |
| particulated milk protein composition | | |
| Total | 100 | 100 |

As illustrated, increasing protein in yogurt applications is challenging. Yogurt's viscosity commonly increases as protein increases. The reason is that proteins such as casein proteins and undenatured whey proteins commonly used to fortify foods bind and interact with water resulting in a higher viscosity texture. A benefit of using co-particulated milk protein compositions according to the present technology in products such as yogurt is to fortify the protein content and maintain acceptable viscosity. As illustrated, the drinkable yogurt fortified with regular-undenatured WP80 has a viscosity of 350 centipoise (cP), which is too viscous to be acceptable for a drinkable yogurt product. On the other hand, yogurts fortified with microparticulated or coparticulated milk protein compositions according to the present technology exhibited an acceptable viscosity of 36-104 cP for pourability and as a drinkable yogurt product. In addition, the co-particulated milk protein compositions exhibited greatly preferred performance as compared to the commercial reference at a 350 cP viscosity.

### Example 6

### Spoonable Yogurt

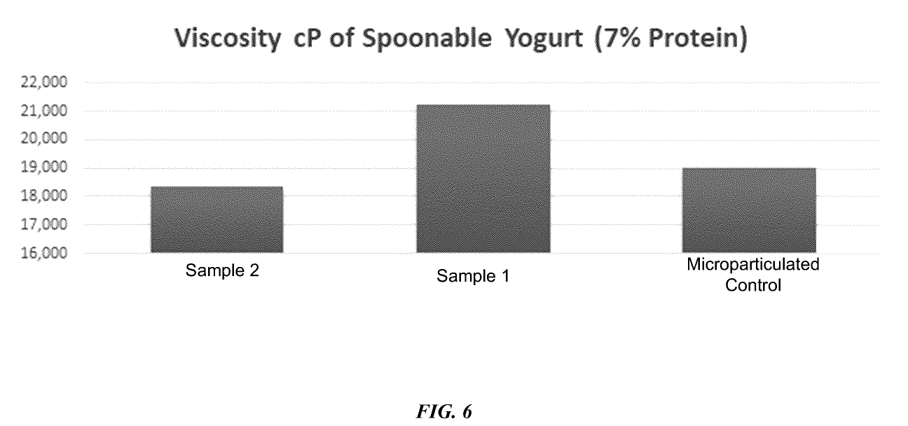

Table 4 and FIG. 6 show examples of protein fortified spoonable yogurt. The data illustrates that microparticulated and co-particulated milk protein compositions according to the present disclosure exhibit higher leverage in increasing protein content of a spoonable yogurt without significant increase in viscosity or changes in acceptable texture. The spoonable yogurt base with 7% microparticulated control protein had a viscosity of 19,000 cP. When protein is increased with co-particulated protein compositions according to the present technology, the spoonable yogurt viscosities were equivalent to the commercial reference yogurt at similar % protein. This demonstrates the co-particulated composition according to the present technology can be utilized to achieve an increase in protein content of spoonable yogurt while maintaining viscosity. Such improvements also improve the consumer experience due to the more familiar viscosity and texture.

TABLE 4

| | Spoonable Yogurt base Recipes | |
|---|---|---|
| | 7% Protein after fortification | |
| | w/mpWP or cpMilk-protein | |
| | W/W % in | W/W % range in |
| Ingredients | base recipe | base recipe |
| Skim milk | 0 | 0 |
| Ultrafiltered Milk | 97.5 | 97-98 |
| Microparticulated control or Co- | 2.5 | 2.0-3.0 |
| particulated milk protein composition | | |
| Total | 100 | 100 |

### Example 7 Degree of Thermal Aggregation

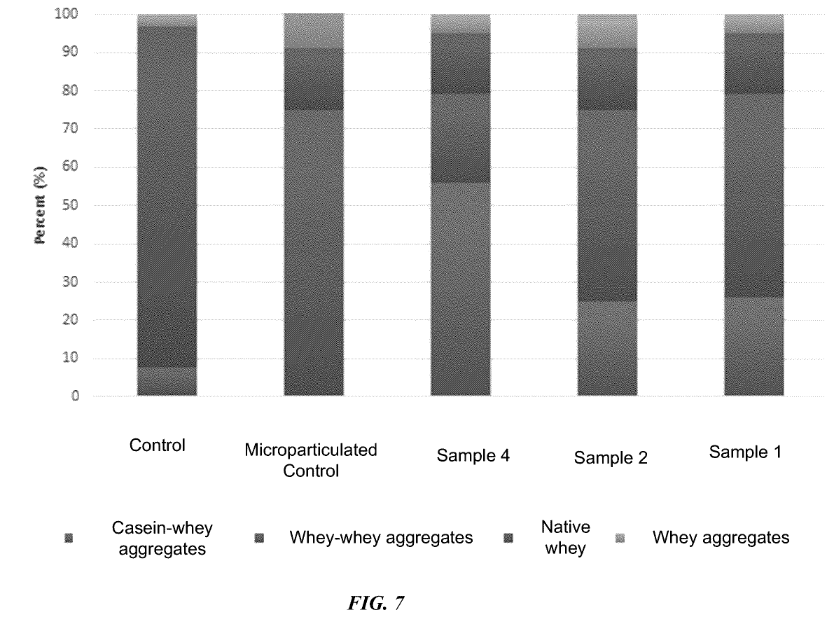

Co-particulated milk proteins, microparticulated whey protein (microparticulated control), and regular whey protein (control) will be evaluated for the degree of thermal aggregation by treating solution of each protein composition with both an acid precipitation method and rennet precipitation method followed by centrifugation and evaluating the supernatant and pellets by total Kjeldahl protein analysis and capillary electrophoresis to mass balance the amount of each protein pool. FIG. 7 depicts the anticipated distribution of thermally aggregated products in regular, microparticulated and co-particulated protein products. Because casein is not present the regular whey protein (control) and microparticulated whey protein (microparticulated control) do not contain casein-whey aggregates. The control has not undergone the thermal treatment described in Example 1 and exhibits relatively small amounts of insoluble protein particles. The amount of insoluble protein particles in the microparticulated control would have been created through standard milk and whey pasteurization and spray drying. FIG. 7 illustrates that co-particulated milk proteins have a lower percentage of whey-whey aggregates which is significantly different from the reference product microparticulated whey protein (microparticulated control). Also indicative of co-particulated milk proteins is the significant presence of casein-whey aggregates. The presence of casein-whey aggregates is an indication of the interaction between the casein and whey proteins. As the amount of casein increases in the case of Sample 4, the percentage of casein-whey aggregates increase with a resulting decrease in the percentage of whey-whey aggregates.

Casein micelles range in size between 0.1 to 0.6 microns and due to casein's high thermal stability would not denature alone during the thermal treatment described in Example 1. Due to the thermal treatment described in Example 1, however, whey protein denatures and interacts with cysteine residue-containing casein micelles through thiol group disulfide exchange reactions causing casein-whey protein aggregates. Following thermal treatment, many whey proteins are either present in whey-whey aggregates or covalently associated with casein as casein-whey aggregates while the amounts of native whey and whey aggregates remain relatively constant, though there are slight changes that are likely the result of small shift in pH of the material being thermally treated. Owing to the natural size of casein micelles the casein-whey aggregates remain smaller than whey-whey aggregates. The ability of casein-whey aggregates to grow is limited by how much whey protein can be denatured and covalently associated to the casein through disulfide interactions. Interaction between whey protein and casein micelles are limited to κ-casein on the surface of micelle and αs2-casein located in the center of the micelles and more difficult for whey proteins to interact with. This interaction between casein and whey proteins provides a benefit in controlling the viscosity of yogurt with high protein targets per serving by minimizing further interaction of the whey protein-casein protein agglomerates in the processing of a prepared yogurt mix.

### Example 8

### Interaction Between Whey Protein and Casein Micelles

Different blends (90:10, 30:70 & 70:30) of Whey Protein (WP): Casein (CN) on a true protein basis of process feed and their coparticulated products were evaluated for the interaction between whey protein and casein using a rennet treatment method outlined below:
With deionized (DI) water dilute and standardize samples to 5-percent total protein solution (50 g each).
On a stir plate, add rennet (Chymax Ultra, Chr Hansen) proportional to casein in the WP:CN blend (see below) while stirring and stir for 10 seconds.
   a. 3 μL rennet in 90:10 blend
   b. 9 μL rennet in 70:30 blend
   c. 27 μL rennet in 30:70 blend
Place the renneted solution in a 32° C. water bath and incubate for 30 minutes.
Deactivate the rennet by microwave heating the rennet treated samples to boiling point.
Run capillary electrophoresis (CE) analysis (refer to Appendix for CE method) to determine residual kappa-casein in the rennet treated samples.

The blends of WP:CN feed is composed of whey proteins and casein proteins (casein micelles). Kappa-casein in casein micelles is believed to interact with whey protein via covalent disulfide-crosslinking forming casein-whey aggregates during thermal treatment (coparticulation process) described in Example 1 and become inaccessible to rennet cleavage. Based on measuring the difference in residual kappa-casein between rennet treated feed and its coparticulated sample, the aggregated casein due to coparticulation process (CP process), is defined as WP:CN Coparticulation Aggregation Index (CP Index), and is determined as follows:

$$a = b - c$$

Where:
a=WP:CN Coparticulation Aggregation Index (CP Index)
b=residual kappa-casein in rennet treated coparticulated sample
c=residual kappa-casein in rennet treated feed sample

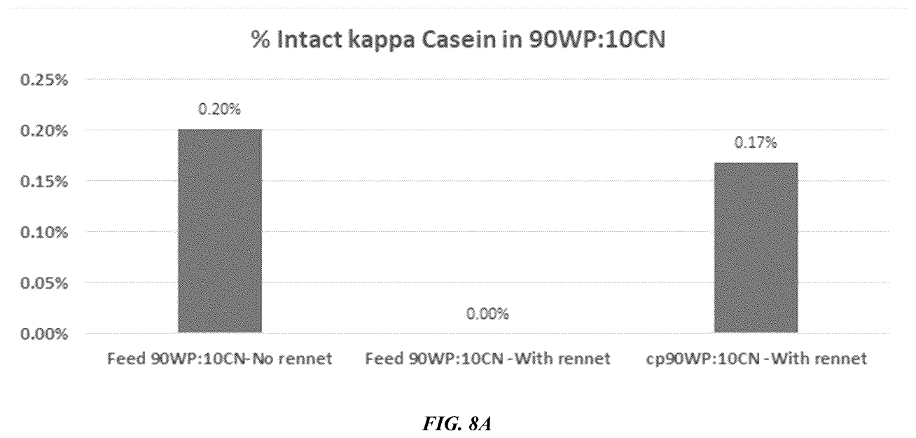

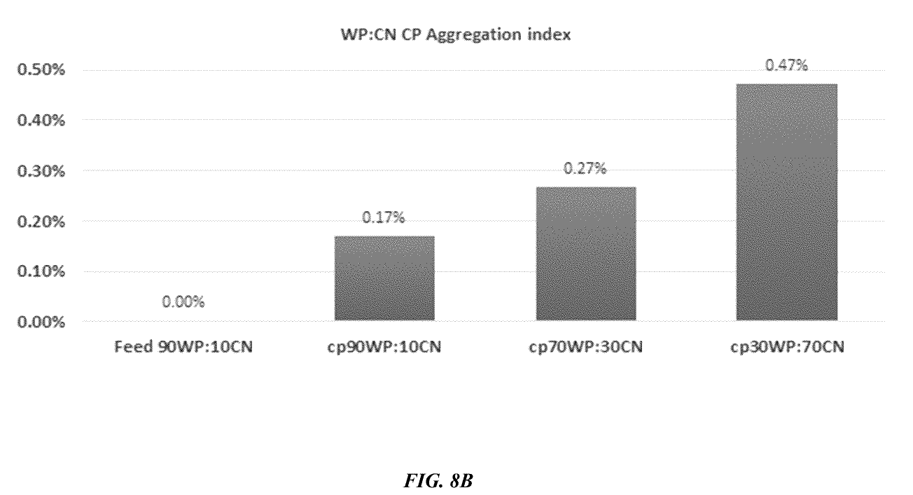

FIG. 8A shows the percent kappa-casein in 90WP:10CN feed before and after rennet treatment, and rennet-treated coparticulated sample cp90WP:10CN. After rennet treatment, the residual kappa-casein of Feed 90WP:10CN became 0%, whereas there is 0.17% of residual kappa-casein in the rennet-treated cp90WP:10CN sample. Moreover, the CP Index of cp90WP:10CN is calculated to be 0.17% using the above equation.

Casein would not denature during the thermal treatment (CP process) described in Example 1. However, whey proteins, especially beta-lactoglobulin (beta-lg), can engage in interactions with casein micelles through thiol group-disulfide bridge exchange reactions with kappa-casein, limiting its access and cleavage by rennet.

As shown in FIG. 8B, the CP Index increased from 0% in Feed 90WP:10CN to 0.47% in cp30WP:70CN, suggesting that interactions between whey protein and casein micelles are attributed to the thermal treatment, and the extent of interaction increased with increased level of casein in the matrix. This explains the dependence of WP:CN aggregation to relative stoichiometric concentration of reactive (thiol group containing) casein components (i.e., kappa-casein) to whey protein.

Following thermal treatment, the casein micelles' “hairy layer” contains whey proteins covalently associated with casein as casein-whey aggregates. Interaction between whey protein and casein micelles are limited to kappa-casein on the surface of micelle and alpha-s2-casein located in the center of the micelles. This interaction between whey protein and casein provides a benefit in controlling the viscosity of yogurt with high protein targets per serving by minimizing further interaction of the WP-CN agglomerates in the processing of a prepared yogurt mix.

As used herein, the terms “about” or “approximately” or “substantially” may be interpreted as being within a range that would be expected by one having ordinary skill in the art in light of the specification.

In the foregoing description, for the purposes of explanation, numerous specific details were set forth in order to provide a thorough understanding of various embodiments. It will be apparent, however, that some embodiments may be practiced without some of these specific details. In other instances, well-known structures and devices are shown in block diagram form.

The foregoing description provides exemplary embodiments only, and is not intended to limit the scope, applicability, or configuration of the disclosure. Rather, the foregoing description of various embodiments will provide an enabling disclosure for implementing at least one embodiment. It should be understood that various changes may be made in the function and arrangement of elements without departing from the spirit and scope of some embodiments as set forth in the appended claims.

Also, it is noted that individual embodiments may have beeen described as a process which is depicted as a flowchart, a flow diagram, a data flow diagram, a structure diagram, or a block diagram. Although a flowchart may have described the operations as a sequential process, many of the operations can be performed in parallel or concurrently. In addition, the order of the operations may be re-arranged. A process is terminated when its operations are completed, but could have additional steps not included in a figure. A process may correspond to a method, a function, a procedure, a subroutine, a subprogram, etc. When a process corresponds to a function, its termination can correspond to a return of the function to the calling function or the main function.

In the foregoing specification, features are described with reference to specific embodiments thereof, but it should be recognized that not all embodiments are limited thereto. Various features and aspects of some embodiments may be used individually or jointly. Further, embodiments can be utilized in any number of environments and applications beyond those described herein without departing from the broader spirit and scope of the specification. The specification and drawings are, accordingly, to be regarded as illustrative rather than restrictive.

1. A co-particulated milk protein composition comprising:
at least 60 wt. % total protein on a dry weight basis, based upon the weight of the co-particulated milk protein composition;
greater than about 65 wt. % whey protein based on a weight of total true protein;
from about 5 wt. % to about 35 wt. % casein protein based on the weight of total true protein; and
wherein at least a portion of the total protein comprises casein-whey protein complexes and whey-whey protein complexes, and wherein the co-particulated milk protein composition exhibits a co-particulation index of greater than zero to less than or about 0.3%.

2. The co-particulated milk protein composition according to claim 1, wherein the co-particulated milk protein composition includes milk protein particles, and wherein milk protein particles having a particle size of 1 to 10 μm form less than 50 wt. % of the total true protein, and/or wherein milk protein particles having a particle size of less than 0.5 μm form greater than or about 30 wt. % of the total true protein.

3. The co-particulated milk protein composition according to claim 1, wherein the co-particulated milk protein composition includes milk protein particles, wherein milk protein particles comprise a D90 particle size distribution value of about 5 μm or less.

4. The co-particulated milk protein composition according to claim 1, wherein the co-particulated milk protein composition comprises less than or about 10 wt. % fat on a dry weight basis, based upon the weight of the co-particulated milk protein composition.

5. The co-particulated milk protein composition according to claim 1, wherein the whey protein and the casein protein are present in the co-particulated milk protein composition at a ratio of whey protein to micellar casein protein of about 60:40 to about 95:5.

6. The co-particulated milk protein composition according to claim 1, wherein the casein-whey protein complexes comprise one or more covalent disulfide bonds.

7. A co-particulated milk protein composition comprising:
at least 60 wt. % total protein on a dry weight basis, based upon the weight of the co-particulated milk protein composition;
greater than about 60 wt. % whey protein based on a weight of total true protein;
from about 5 wt. % to about 40 wt. % casein protein based on the weight of total true protein; and
wherein at least a portion of the total protein comprises casein-whey protein complexes and whey-whey protein complexes, the co-particulated milk protein composition includes milk protein particles having a particle size of 1 to 10 μm and a D50 particle size of about 5 μm or less, and wherein the milk protein particles having a particle size of 1 to 10 μm form less than 50 wt. % of the total true protein_.

8. The co-particulated milk protein composition according to claim 7, wherein the co-particulated milk protein composition includes milk protein particles, wherein milk protein particles having a particle size of less than 0.5 μm form greater than or about 30 wt. % of the total true protein.

9. The co-particulated milk protein composition according to claim 7, wherein milk protein particles having a particle size of 1 to 10 μm form less than 45 wt. % of the total true protein.

10. The co-particulated milk protein composition according to claim 7, wherein milk protein particles having a particle size of 1 to 10 μm form less than 40 wt. % of the total true protein.

11. The co-particulated milk protein composition according to claim 7, wherein the co-particulated milk protein composition comprises greater than or about 7 wt. % alpha-lactalbumin on a dry weight basis, based on the weight of total true protein.

12. A fortified food and/or beverage product, comprising:
greater than or about 4 wt. % protein, based upon a weight of the food and/or beverage product;
wherein the fortified food and/or beverage product comprises a co-particulated milk protein composition according to claim 1.

13. The fortified food and/or beverage product of claim 12, wherein the fortified food and/or beverage product comprises a spoonable or drinkable yoghurt.

14. A fortified food and/or beverage product, comprising:
greater than or about 4 wt. % protein, based upon a weight of the food and/or beverage product;
wherein the fortified food and/or beverage product comprises a co-particulated milk protein composition according to claim 2.

15. The fortified food and/or beverage product of claim 14, wherein the fortified food and/or beverage product comprises a spoonable or drinkable yoghurt.

16. A method of making a co-particulated milk protein composition, the method comprising:
forming a mixture of a whey protein composition and a casein source at a ratio of about 60:40 to about 95:5;
denaturing the mixture at a temperature of greater than or about 70° C. and a pH of about 6 to 6.8, forming co-particulated milk protein particles;
reducing a particle size of the co-particulated milk protein particles in the mixture, thereby forming the co-particulated milk protein composition, and
wherein at least a portion of total protein comprises casein-whey protein complexes and whey-whey protein complexes, the co-particulated milk protein composition comprises at least 60 wt. % total protein on a dry weight basis, based upon the weight of the co-particulated milk protein composition, the co-particulated milk protein particles include milk protein particles having a particle size of 1 to 10 μm, and wherein the milk protein particles having a particle size of 1 to 10 μm form less than 50 wt. % of total true protein.

17. The method of claim 16, wherein the casein source comprises micellar casein concentrate, micellar casein isolate, ultrafiltered milk protein, microfiltered milk protein, milk protein concentrate, milk protein isolate, a caseinate, a caseinate salt, rennet casein, non-animal casein, acid casein, or a combination thereof.

18. The method of claim 16, wherein the whey protein composition comprises whey protein concentrate, whey protein isolate, native whey protein concentrate, native whey protein isolate, acid whey protein, or a combination thereof.

19. The method of claim 16, wherein denaturing comprises forming one or more covalent disulfide bonds between a casein and a whey protein.

20. A fortified food and/or beverage product formed according to the method of claim 16.
